(19) United States
(12) Patent Application Publication
(10) Pub. No.: US 2015/0366825 A1
(43) Pub. Date: Dec. 24, 2015

(54) MULTIPARTICULATE PHARMACEUTICAL COMPOSITION COMPRISING A MULTITUDE OF TWO KINDS OF PELLETS

(71) Applicants: Shraddha Sanjeev JOSHI, Navi Mumbai (IN); Ashish Sharadchandra GUHA, Mumbai Dombivali (E) (IN); Suresh Shahuraj DOKE, Navi Mumbai, Maharashtra (IN); Ashishkumar Prafulchandra SHAH, Gujarat (IN); Preeti PATIL, Mumbai, Maharashtra (IN)

(72) Inventors: Shraddha Sanjeev JOSHI, Navi Mumbai (IN); Ashish Sharadchandra GUHA, Mumbai Dombivali (E) (IN); Suresh Shahuraj DOKE, Navi Mumbai, Maharashtra (IN); Ashishkumar Prafulchandra SHAH, Gujarat (IN); Preeti PATIL, Mumbai, Maharashtra (IN)

(73) Assignee: EVONIK ROEHM GMBH, Darmstadt (DE)

(21) Appl. No.: 14/764,804

(22) PCT Filed: Apr. 25, 2013
(86) PCT No.: PCT/EP2013/058625

§ 371 Date: Jul. 30, 2015

(30) Foreign Application Priority Data
Feb. 13, 2013 (IN) 627/CHE/2013

(51) Int. Cl. A61K 31/138; A61K 9/00; A61K 9/48; A61K 9/50; A61K 9/20

(52) U.S. Cl. CPC A61K 31/138; A61K 9/5026; A61K 9/5084; A61K 9/2081; A61K 9/4808; A61K 9/4833; A61K 9/2095; A61K 9/009

(57) ABSTRACT

Multiparticulate pharmaceutical composition comprising a multitude of two kinds of pellets, A and B, each comprising metoprolol or a pharmaceutically acceptable salt thereof as an active pharmaceutical ingredient, wherein the pellets A are coated with a coating layer comprising at least 30% by weight of a polymeric compound consisting of one or more (meth)acrylate copolymers polymerized from 20 to 40% by weight of ethyl acrylate, 60 to 80% by weight of methyl methacrylate and 0 or less than 5% by weight of methacrylic acid or acrylic acid, in an amount sufficient to result in an active pharmaceutical ingredient release profile according to USP in pH 6.8 test medium with a release rate of less than 20% after 4 hours, wherein the pellets B are not coated or coated with a coating layer and show an active pharmaceutical ingredient release profile according to USP in pH 6.8 test medium with an active pharmaceutical ingredient release rate of more than 40% after 4 hours, wherein the metoprolol release rate of the pellets A in pH 1.2 test medium according to USP with the addition of 40% (v/v) ethanol is •not more than 15% after 15 minutes •more than 15 up to 40% after 30 minutes, wherein the metoprolol release rate of the pellets B in pH 1.2 test medium according to USP with the addition of 40% (v/v) ethanol is •more than 15% after 15 minutes •more than 40% after 30 minutes and wherein the pellets A and B are present in the multiparticulate pharmaceutical composition in a relation resulting in a combined active pharmaceutical ingredient release profile of the multiparticulate pharmaceutical composition according to USP in pH 6.8 test medium with releases rates of •not more than 25% after 1 hour •20 to 40% after 4 hours •40 to 60% after 8 hours •not less than 80% after 20 hours.

### FIELD OF THE INVENTION

The invention is concerned with a multiparticulate pharmaceutical composition comprising a multitude of two kinds of pellets comprising metoprolol as active pharmaceutical ingredient

### TECHNICAL BACKGROUND

WO 02/058677A1 describes a new film coating. The film coating composition comprises a) an acrylic polymer dispersion, e.g. an ethylacrylate/methylmethacrylate copolymer such as EUDRAGIT® NE30D, b) surfactant, c) sodium stearyl fumarate and d) a water-containing liquid useful for the achievement of controlled release from pharmaceutical formulations such as tablets, pellets, etc. . . . . The coating levels which may be calculated from examples are below 20% by weight calculated on the pellet cores.

WO 03/051340A1 describes a new film coating. The film coating composition comprises a dispersion which includes a) an acrylic polymer dispersion, e.g. an ethylacrylate/methylmethacrylate copolymer such as EUDRAGIT® NE30D, b) a vinyl acetate polymer such as KOLLICOAT® SR30D c) a water-containing liquid. The film coat is useful for the achievement of modified release from pharmaceutical formulations such as tablets, pellets, etc. . . . . The coating levels which may be calculated from examples range from about 5 to less than 20% by weight calculated on the pellet cores.

WO2004/012718A1 describes a new film coating. The film coating composition comprises a dispersion which includes a) an acrylic polymer, which is EUDRAGIT® NE30D, b) an anti-sticking agent, which is glycerol monostearate (GMS), c) a surface active agent wherein the surface active agent is in an amount less than 1.3% by weight of the dispersion, and d) a water-containing liquid, wherein the dispersion does not contain a vinyl acetate polymer. The coating levels which may be calculated from examples range from about 17 to 25% by weight calculated on the pellet cores.

WO 2008/012346A1 describes an extended release pharmaceutical formulation of metoprolol and process for its preparation. The extended release pharmaceutical formulation comprises extended release coated granules having a particle size from 0.2 to 2 mm, a friability lower or equal to 1% and comprises metoprolol succinate in an amount of 10 to 75% by weight and at least one binder selected from microcrystalline cellulose and methylcellulose, said granule being coated with a film-former agent like for instance ethylcellulose.

WO 2010/105672A1 describes controlled release pharmaceutical composition with resistance against the influence of ethanol employing a coating comprising neutral vinyl copolymers of the EUDRAGIT® NE type. Examples with metoprolol succinate pellets are shown, however the release profiles differ from those disclosed herein.

### Object and Solution

Multiparticulate pharmaceutical compositions comprising a multitude of one type of pellets comprising metoprolol are known. If these kind of extended release pharmaceutical compositions release a large or the entire dose of metoprolol caused accidentally by mechanical influences or the influence of ethanol for instance, this may cause severe problems to the patient. The risk of the so-called “dose-dumping” cannot be totally excluded but should be reduced as much as possible. Thus it was an object of the present application to provide a multiparticulate pharmaceutical composition for metoprolol with a lowered risk of “dose-dumping”. In WO 02/058677A1, WO 03/051340A1 and WO2004/012718A1 the coating levels which may be calculated from examples range from about 5 to 25% by weight calculated on the pellet cores, which may be estimated as from thin to medium. The inventors started from the idea that thicker coatings are usually more resistant to “dose-dumping” than thin or medium coatings. However the increase of the thickness of the coating of the pellets included in a multiparticulate pharmaceutical composition again leads to a more flat release curve which may not match the desired active ingredient release anymore. However when a majority of thick coated pellets are mixed with a minority of low coated or uncoated pellets the desired release profile may be re-established again. The low coated or uncoated pellets are not relevant in respect to “dose-dumping” since they only represent a minority of the total dose and it is intended anyway that this dose shall be release quickly. The majority of the dose however is included in the thick coated pellets with increased resistance against “dose-dumping”. This has the advantage that the risk of “dose-dumping” is overall lowered and can be better controlled.

Another object of the present application is that the release profile required for Metoprolol, presented by the release profile of the present originator product TOPROL®XL, has to be matched at pH 6.8. In the medium pH 1.2 replaced with 40% ethanol v/v, the release profile of the inventive multiparticulate pharmaceutical composition should be lower or comparable according to the definitions given by the US Food and Drug Administration (FDA) in the document “Establishing Bioequivalence of Modified-Release Products: OGD Perspective” by Barbara M. Davit, Ph.D., J. D., Acting Director, Division of Bioequivalence 2, Office of Generic Drugs Center for Drug Evaluation and Research (CDER), United States Food and Drug Administration (US-FDA) (www.aaps.org/Meeting_and..../Past.../Thu_{—}1330_{—}202B_Davit/)

Requirement to produce comparative results of the originator product (RLD) and the product in question (test product) using above alcoholic dissolution medium is recommended by the Office of Generic Drugs (OGD) recommendations (Nonbinding Recommendations/Draft Guidance for Metoprolol Succinate, recommended January 2008, April 2008) to fulfil the requirements for a generic product.

### DETAILED DESCRIPTION OF THE INVENTION

### Multiparticulate Pharmaceutical Composition

The invention discloses a multiparticulate pharmaceutical composition comprising a multitude of two kinds of pellets, A and B (type A and type B), each comprising metoprolol or a pharmaceutically acceptable salt thereof as an active pharmaceutical ingredient.

The pellets A and B each comprise a metoprolol comprising core. The pellets A may comprise a coating, which provides an extended release profile. The pellets B may be uncoated but preferably may comprise a coating which provides a release profile which is faster than that of the pellets A.

A multitude of two kinds of pellets, A and B shall mean that one single unit of the multiparticulate pharmaceutical composition, for instance one tablet, may contain more than 100, more than 1000 pellets A and more than 50, more than 80, more than 100, more than 200, more than 250 pellets B. One unit of the multiparticulate pharmaceutical composition may for instance contain 100 to 5000, 700 to 4000 pellets A and 50 to 2000, 80 to 1500 pellets B.

A multitude of two kinds of pellets, A and B shall mean that one single unit of the multiparticulate pharmaceutical composition, for instance one tablet containing 40-60, for instance 25 mg metoprolol succinate, may contain more than 100, more than 1000 pellets A and more than 50, more than 80, more than 100, more than 200, more than 250 pellets B. One unit of the multiparticulate pharmaceutical composition may for instance contain 100 to 2000, 700 to 1800 pellets A and 50 to 500, 80 to 400 pellets B.

A multitude of two kinds of pellets, A and B shall mean that one single unit of the multiparticulate pharmaceutical composition, for instance one tablet containing 180-220, for instance 200 mg metoprolol succinate, may contain more than 1000, more than 2000 pellets A and more than 100, more than 160, more than 200, more than 400, more than 500 pellets B. One unit of the multiparticulate pharmaceutical composition may for instance contain 1000 to 5000, 1400 to 4000 pellets A and 100 to 2000, 150 to 1500 pellets B.

Comprising two kinds of pellets, A and B, shall mean that the multiparticulate pharmaceutical composition may contain beside pellets of the type A and B, which are within the definitions given herein, also other kinds of active ingredients. Active ingredients, for instance like hydrochlorothiazide, which are not in the form of pellets, for instance included in the compression material in the case of a tablet, or which are alternatively in the form of pellets may be comprised if necessary or useful for pharmaceutical reasons. As a rule only pellets of the types A and B are present in the multiparticulate pharmaceutical composition. In this case the multiparticulate pharmaceutical composition contains two kinds of pellets, A and B. It is possible to include a multitude of different individual types of pellet A and/or a multitude of different individual type pellet B in the multiparticulate pharmaceutical composition. Preferably a multitude of only one individual type of pellet A and a multitude of one individual type pellet B is present in the multiparticulate pharmaceutical composition.

The multiparticulate pharmaceutical composition may comprise, contain or include an amount of pellets A and B which is at least 10, at least 20, at least 30, at least 40, at least 50, at least 60, at least 70, at least 80% by weight. The multiparticulate pharmaceutical composition may comprise, contain or include an amount of pellets A and B which is from 10 to 90, preferably from 20 to 60% by weight.

The multiparticulate pharmaceutical composition may comprise beside pellets A and B pharmaceutical excipients like for instance celluloses functioning as processing aids for instance for preparing pellet A and B containing compressed tablets.

Pellets A and pellets B may independently from each other comprise 20 to 80, 30 to 70 or 35 to 65% metoprolol by weight. Pellets A and pellets B may comprise the same absolute amount of metoprolol. Preferably pellets A and pellets B may have identical cores. Preferably pellets A and pellets B may have identical coatings but differ in the thickness of the coating applied.

### Pellets A/Polymeric Compound

The pellets A are comprising, essentially consisting of or consisting of a core comprising metoprolol and a coating, preferably an extended release coating, applied to the core.

The pellets A may be coated with a coating layer comprising at least 30, at least 40, at least 50, at least 60 at least 70 at least 80, at least 90% by weight or up to 100% of a polymeric compound consisting of one or more (meth)acrylate copolymers polymerized from 20 to 40% by weight of ethyl acrylate, 60 to 80% by weight of methyl methacrylate and 0 or less than 5% by weight of methacrylic acid or acrylic acid (polymer type EUDRAGIT® NE or EUDRAGIT® NM). The coating may be applied in an amount sufficient to result in an active pharmaceutical ingredient release profile according to USP in pH 6.8 test medium with a release rate of less than 20, less than 15 less than 10 less than 5% after 4 hours. The release rate may be from 2 to less than 20%.

The pellets A may be equipped with a coating layer comprising 30 to 100, 35 to 90 preferably 40 to 80% by weight of a polymeric compound consisting of one or more (meth)acrylate copolymers polymerized from 20 to 40% by weight of ethyl acrylate, 60 to 80% by weight of methyl methacrylate and 0 or less than 5% by weight of methacrylic acid or acrylic acid (polymer type EUDRAGIT® NE or EUDRAGIT® NM).

The Pellets A may comprise a coating layer comprising the polymeric compound in an amount of at least 28, of at least 30, of at least 35, of at least 40% by weight based on the weight of the pellet cores. The Pellets A may be coated with a coating layer comprising the polymeric compound in an amount of at least 28 to 50, 30 to 45, 35 to 44% by weight based on the weight of the pellet cores.

### Pellets B

The pellets B are comprising, essentially consisting of or consisting of a core comprising metoprolol and a coating, preferably an extended release coating, applied to the core.

The pellets B may be not coated or coated with a coating layer and show an active pharmaceutical ingredient release profile according to USP in pH 6.8 test medium with an active pharmaceutical ingredient release rate of more than 40, more than 50 more than 60 more than 70% after 4 hours. The release rate pH 6.8 after 4 hours may be from more than 40 up to 100%.

The pellets B may be coated with a coating layer comprising at least 30, at least 40, at least 50, at least 60 at least 70 at least 80, at least 90% by weight or up to 100% of a polymeric compound consisting of one or more (meth)acrylate copolymers polymerized from 20 to 40% by weight of ethyl acrylate, 60 to 80% by weight of methyl methacrylate and 0 or less than 5% by weight of methacrylic acid or acrylic acid (polymer type EUDRAGIT® NE or EUDRAGIT® NM). The coating may be applied in an amount sufficient to result in an active pharmaceutical ingredient release profile according to USP in pH 6.8 test medium with a release rate of more than 40, more than 50, more than 60% after 4 hours. The release rate may be from more than 40 to 60%.

The Pellets B may comprise a coating layer comprising the polymeric compound in an amount of less than 28, not more than 25, not more than 20, not more than 15, not more than 12% by weight calculated on the weight of the pellet cores. The Pellets B may comprise a coating layer comprising the polymeric compound in an amount of 5 to less than 28, 5 to 25 or 5 to 15, preferably 8 to 12% by weight based on the weight of the pellet cores.

### Metoprolol Release Rate in pH 6.8 Test Medium

The pellets A and B may be present in the multiparticulate pharmaceutical composition in a relation resulting in a combined active pharmaceutical ingredient release profile of the multiparticulate pharmaceutical composition according to USP (USP=United States Pharmacopeia, for instance USP32) in pH 6.8 test medium with releases rates of
not more than 25% after 1 hour
20 to 40% after 4 hours
40 to 60% after 8 hours
not less than 80% after 20 hours.

The relation in which the pellets A and B depends may be mixed depends on their individual composition, e.g. metoprolol content and coating thickness, respectively their individual release profiles.

The individual release profiles of pellets A depends on the composition of the core and on the certain kind of coatings and on the coating levels (thickness) applied.

The individual release profile of pellets B depends on the composition of the core alone if the pellets B are uncoated. If the pellets B are coated, the individual release profile of pellets B depends on the composition of the core and on the certain kind of coatings and on the coating levels (thickness) applied.

At last the relation in which the pellets A and B depends may be mixed depends also on the overall formulation of the multiparticulate pharmaceutical composition.

All these influences have to be combined to end up with the pH 6.8 metoprolol release profile as shown above.

### Mixture of Pellets A and Pellets B

Preferably the pH 6.8 metoprolol release profile as shown above may be achieved when the multiparticulate pharmaceutical may comprise an amount of at least 65, at least 80, at least 90, at least 95% by weight of the active ingredient in the form of pellets A (as pellets A) and not more than 35, not more than 20 not more than 10, not more than 5% by weight of the active ingredient in the form of pellets B (as pellets B). The active ingredient in the form of pellets A and pellets B may add up in total to 100%

The pellets A and B present in the multiparticulate pharmaceutical composition may show an arithmetically mean value of the coating layers calculated on the total weight of the pellets of 25 to 50, 35-45% by weight.

In some cases the release profile of the pure pellet A and B mixture may slightly differ from that of the final multiparticulate pharmaceutical composition. For instance the 4 h pH 6.8 release value may be lower for the pure pellet A and B mixture than for the final multiparticulate pharmaceutical composition, for instance a compressed tablet, containing that certain pellet mixture. This may be due to the influence of further pharmaceutical excipients included or to the processing conditions applied.

### Cores

Pellets A and B each comprise a metoprolol comprising core. Metoprolol may be present in the core in amounts of 50 to 95, preferably 55 to 90% by weight.

The cores are preferably generated by extrusion spheronization and may further comprise pharmaceutical acceptable excipients, preferably but not limited to celluloses, like for instance microcrystalline cellulose, hydroxy propyl cellulose, sodium carboxy methyl cellulose or hydroxy propyl methyl cellulose, in amounts of 5 to 50, preferably 10 to 45% by weight.

### Coatings

Pellets A and/or optionally pellets B may be coated with a coating layer comprising one or more (meth)acrylate copolymers polymerized from 20 to 40% by weight of ethyl acrylate, 60 to 80% by weight of methyl methacrylate and 0 or less than 5%, preferably not more than 2% by weight, more preferably not more than 1 or 0.05 to 1% by weight of methacrylic acid or acrylic acid (EUDRAGIT® NE or EUDRAGIT® NM polymer type).

Suitable are EUDRAGIT® NE and Eudragit® NM which are copolymers composed of free-radically polymerized units of 30% by weight of ethyl acrylate and 70% by weight of methyl methacrylate.

Preference is given to neutral or essentially neutral methyl acrylate copolymers which, according to WO 01/68767, have been prepared as dispersions using 1-10% by weight of a nonionic emulsifier having an HLB value of 15.2 to 17.3. The latter offer the advantage that there is no phase separation with formation of crystal structures by the emulsifier (Eudragit® NM type).

According to EP 1 571 164 A2, corresponding or similar, virtually neutral (meth)acrylate copolymers with small proportions of 0.05 to 1% by weight of monoolefinically unsaturated C3-C8-carboxylic acids can be prepared by emulsion polymerization in the presence of comparatively small amounts of anionic emulsifiers, for example 0.001 to 1% by weight.

Since for the pellets B a comparatively rapid release type has to be realized, pellets B need not to be coated or could be coated by fast dissolving coatings as well. However, preferably the coatings of pellets B are of the same type as those from pellets A but are applied in lower thickness or coat build-up.

Beside the polymer the coatings for pellets A and optionally for pellets B may further comprise pharmaceutical acceptable excipients, preferably but not limited to celluloses, like for instance microcrystalline cellulose, hydroxy propyl cellulose or hydroxy methyl propyl cellulose, glidants like talc. Suitable amounts may be up to 70%, 10 to 65, preferably 20 to 60% by weight based on the total weight of the coating. Celluloses, like for instance microcrystalline cellulose, hydroxy propyl cellulose, sodium carboxy methyl cellulose or hydroxy propyl methyl cellulose may be included in amounts of 5 to 25% by weight. Glidants like talc may be included in amounts of 20 to 50% by weight.

The Pellets A may comprise a coating layer comprising the polymeric compound as disclosed above in an amount of at least 28, of at least 30, of at least 35, of at least 40% by weight based on the weight of the pellet cores. The Pellets A may be coated with a coating layer comprising the polymeric compound in an amount of at least 28 to 50, 30 to 45, 35 to 44% by weight based on the weight of the pellet cores.

The Pellets B may comprise a coating layer comprising the polymeric compound as disclosed above in an amount of less than 28, not more than 25, not more than 20, not more than 15, not more than 12% by weight calculated on the weight of the pellet cores. The Pellets B may comprise a coating layer comprising the polymeric compound in an amount of 5 to less than 28, 5 to 25 or 5 to 15, preferably 8 to 12% by weight based on the weight of the pellet cores.

Metoprolol Release Rate in pH 1.2 Test Medium with 40% (v/v) Ethanol

Another object of the present application is that the release profile required for metoprolol, presented by the release profile of the present originator product TOPROL®XL, has to be matched at pH 6.8. In the medium pH 1.2 replaced with 40% ethanol v/v, the release profile of the inventive multiparticulate pharmaceutical composition should be lower or comparable according to the definitions given by the US Food and Drug Administration (FDA) in the document “Establishing Bioequivalence of Modified-Release Products: OGD Perspective” by Barbara M. Davit, Ph.D., J. D., Acting Director, Division of Bioequivalence 2, Office of Generic Drugs Center for Drug Evaluation and Research (CDER), United States Food and Drug Administration (US-FDA) (www.aaps.org/Meeting_and..../Past.../Thu_{—}1330_{—}202 B_Davit/)

Requirement to produce comparative results of the originator product (RLD) and the product in question (test product) using above alcoholic dissolution medium is recommended by the Office of Generic Drugs (OGD) recommendations (Nonbinding Recommendations/Draft Guidance for Metoprolol Succinate, recommended January 2008, April 2008) to fulfil the requirements for a generic product.

The metoprolol release rate of the pellets A in pH 1.2 test medium according to USP (for instance USP 32) containing 40% (v/v) ethanol may be
not more than 15% or 2 to 12% after 15 minutes
more than 15 up to 40% or 25 to 40% after 30 minutes,

The metoprolol release rate of the pellets B in pH 1.2 test medium according to USP (for instance USP 32) containing 40% (v/v) ethanol may be
more than 15% or more than 15 up to 100% after 15 minutes
more than 40% or more than 40 up to 100% after 30 minutes

The Unites States Food and Drug Administration (FDA) recommends that an extended release pharmaceutical formulation like for instance those for metoprolol shall be robust against the influence of ethanol, especially under test condition according to USP pH 1.2 for 2 hours modified with the addition of ethanol to a final concentration of 40% (v/v).

A generic product shall have at least a lower or a comparable release curve in comparison to the originator or innovator product. If the release curve of the generic product is lower than the release curve of the originator product the similarity factor (f2-value) generated from the pair wise comparison of the release values from 15 min to 2 hours in 15 minutes intervals may be less than 50. If the release curve is comparable the similarity factor (f2-value) may be 50 or more than 50.

The calculation of the similarity factor f2 is well known to a skilled person in the field of pharmacy or galenics. The similarity factor f2 may be calculated as described in “Guidance for Industry: Dissolution Testing of Immediate Release Solid Oral Dosage Forms. U.S Department of Health and Human Services, Food and Drug Administration, Center for Drug Evaluation and Research (CDER), August 1997”.

The similarity factor (f2) is a logarithmic reciprocal square root transformation of the sum 2 of squared error and is a measurement of the similarity in the percent (%) dissolution between the two curves.

Preferably the metoprolol release rate of the multiparticulate pharmaceutical composition according to USP in pH 1.2 test medium according to USP (for instance USP 32) for 2 hours with 40% (v/v) ethanol compared to release rate of a known multiparticulate metoprolol composition with only one type of pellets included and with the same metoprolol release profile as shown above has a similarity factor (f2-value) of more than 50.

### A Known Multiparticulate Metoprolol Composition

A known multiparticulate metoprolol composition is a composition which belongs to the state of art in the sense of for instance Article 54 (2) of the European Patent Convention (1973 or 2000). The known multiparticulate metoprolol composition is different from the inventive multiparticulate pharmaceutical composition.

The known multiparticulate metoprolol composition is different from the inventive multiparticulate pharmaceutical composition in for instance in that it has only one type of pellets included.

The known multiparticulate metoprolol composition may be in the form of a compressed metoprolol succinate tablet.

The known multiparticulate metoprolol composition may be an originator medical product.

The known multiparticulate metoprolol composition may be approved for medical use at least in the territory of the United States of America.

TOPROL® XL is an extended release metoprolol succinate tablet product marketed by the company Astra Zeneca under this brand name in the USA. The known multiparticulate metoprolol composition may be TOPROL® XL or a comparable product marketed under a different brand name.

### Originator and Generic

The inventive multiparticulate pharmaceutical composition may be a generic medical product and the known multiparticulate metoprolol composition may be an originator or innovator medical product.

### Multiparticulate Pharmaceutical Composition

The Multiparticulate pharmaceutical composition may be a compressed tablet, a sachet or a capsule.

### Metoprolol

Metoprolol is a selective β₁ receptor blocker used in treatment of several diseases of the cardiovascular system, especially hypertension. Metoprolol succinate is used for extended-release formulations. Metoprolol succinate is currently marketed under brandnames like for instance LOPRESSOR®, TOPROL® XL, Betaloc® ZOK or Beloc® ZOK.

The metoprolol may be present in the inventive multiparticulate pharmaceutical composition in an amount of 5 to 75, 10 to 60 or 15 to 50% by weight.

Pellets A and pellets B may independently from each other comprise 20 to 80, 30 to 70 or 40 to 60% metoprolol by weight. Pellets A and pellets B may comprise the same absolute amount of metoprolol. Preferably pellets A and pellets B may have identical cores and may differ only in their coating thickness.

The term metoprolol as used herein shall include metoprolol salts. Metoprolol salt may be a benzoate, fumarate, succinate or tartrate salt. Metoprolol succinate is the preferred.

### Pharmaceutically Acceptable Excipients

Pharmaceutical acceptable excipients may be used for the cores of the pellets, in the coatings of the pellets and for the formulation of the final multiparticulate pharmaceutical composition.

The pellets A may comprise up to 90, up to 70, up to 50, up to 30, up to 20, up to 10% by weight of pharmaceutically acceptable excipients. The pellets A may comprise 1 to 70, 1 to 50, 5 to 40 or 10 to 30% pharmaceutically acceptable excipients.

The pellets B may comprise up to 90, up to 70, up to 50, up to 30, up to 20, up to 10% by weight of pharmaceutically acceptable excipients. The pellets B may comprise 1 to 70, 1 to 50, 5 to 40 or 10 to 30% pharmaceutically acceptable excipients.

Except from the metoprolol containing pellets, the multiparticulate pharmaceutical composition may comprise up to 90, up to 75 or up to 30% by weight of pharmaceutically acceptable excipients, calculated on the total weight of the multiparticulate pharmaceutical composition. Except from the metoprolol containing pellets, the multiparticulate pharmaceutical composition may comprise 10 to 90, 40 to 80 or 50 to 80% by weight of pharmaceutically acceptable excipients, calculated on the total weight of the multiparticulate pharmaceutical composition.

Pharmaceutical acceptable excipients may be contained for practical reasons, for instance to avoid stickiness or to add a colour. They may be used as processing adjuvants and are intended to ensure a reliable and reproducible preparation process as well as good long-term storage stability, or they achieve additional advantageous properties in the pharmaceutical form. They are added to the polymer formulations before processing and can influence the permeability of the coatings. This property can be used if necessary as an additional control parameter. Of course all kind of excipients used must of course be toxicologically safe and to be used in cosmetics, nutraceuticals or pharmaceuticals without risk for customers or patients.

Pharmaceutical acceptable excipients may comprise antioxidants, brighteners, binding agents, flavouring agents, flow aids, fragrances, glidants, penetration-promoting agents, pigments, plasticizers, polymers, pore-forming agents or stabilizers. The multiparticulate pharmaceutical composition may comprise pharmaceutical acceptable excipients which are celluloses, like for instance microcrystalline cellulose, hydroxy propyl cellulose or hydroxy methyl propyl cellulose, in amounts of 20 to 80 or 30 to 65% by weight. Except from celluloses the multiparticulate pharmaceutical composition may comprise pharmaceutical acceptable excipients, for instance antioxidants, brighteners, binding agents, flavouring agents, flow aids, fragrances, glidants, penetration-promoting agents, pigments, plasticizers, pore-forming agents or stabilizers in amounts of not more than 20 or not more than 10% by weight.

### Process

The application further discloses a process for preparing a multiparticulate pharmaceutical composition according to any preceding claim, by extrusion and spheronisation of active pharmaceutical ingredient containing uncoated pre-pellets for pellets A and B, coating of at least the uncoated pre-pellets A and optionally pre-pellets B by spray coating, formulating pellets A and pellets B as the final multiparticulate pharmaceutical compositions by compression into tablets, by formulation of sachets or by filling into capsules.

### Embodiments which May be Excluded

Preferably the coatings of pellets A and/or B do not contain sodium stearyl fumarate.

Preferably the multiparticulate pharmaceutical composition does not contain glycerol monostearate. Preferably the coatings of pellets A and/or B do not contain glycerol monostearate

Preferably the pellet cores of pellets A and/or B do not contain SiO₂.

Preferably the coatings of pellets A and/or B do not contain ethyl cellulose

### EXAMPLES

### Preparation of Pellets and Tablets

Pellets and Tablets: Metoprolol succinate containing pellet cores (pre-pellets) were produced by extrusion spheronization. The pellet cores were coated by spray coating to final pellets A (43% coating based on EUDRAGIT® NE content by weight of the core) or pellets B (10% coating based on EUDRAGIT® NE content by weight of the core). Pellets were blended in the given ratios together with pharmaceutical excipients as shown below and were compressed to extended release tablets (ER Tablet) according to the quantitative formula below. Pellets with 15, 20, 25, 30 and 40% coatings were produced accordingly.

### Example-5C

### Quantitative Formula for 200 mg Metoprolol Succinate ER Tablets

| | | |
|---|---|---|
| Ingredients | Quantity (mg/tablet) | % w/w |

Composition of the cores

| | | |
|---|---|---|
| Metoprolol succinate | 190 | 24.0 |
| Avicel 101 | 31.67 | 4.0 |
| Avicel CL 611 | 31.67 | 4.0 |
| Total core pellets | 253.34 | 32.0 |

Composition of the coating

| | Pellets B | Pellets A | |
|---|---|---|---|
| | (10% coated) | (43% coated) | |
| Core pellets | 17.73 | 235.61 | 32.0 |
| Talc | 0.89 | 50.66 | 6.5 |
| HPC LF | 0.21 | 12.16 | 1.5 |
| Eudragit NE 30 D | 1.77 | 101.31 | 13.0 |
| (dry polymer) | | | |
| Pellets quantity(mg) | 20.6 | 399.74 | 53.4 |
| | | | (2.6 + 50.4) |
| Pellets quantity (number) | 300 to 500 | 3300 to 4500 | |

Composition of the tablets (Blending and compression)

| | | |
|---|---|---|
| Pellets (A + B) | 420.34 | 53.0 |
| Avicel granules | 81 | 10.2 |
| Avicel 102 | 49.96 | 6.3 |
| Avicel 200 | 101 | 12.7 |
| Ceolus 802 | 92.7 | 11.7 |
| Ac-Di-Sol | 16 | 2.0 |
| Aerosil 200 | 5 | 0.6 |
| Sodium stearyl fumarate | 4 | 0.5 |
| Core tablet weight | 770.00 | 97.1 |

Top Coating Composition

| | | |
|---|---|---|
| HPMC 6 cps | 16.5 | 2.1 |
| Tio2 | 4.95 | 0.6 |
| PEG 6000 | 1.65 | 0.2 |
| Final tablet weight | 793.10 | 100.00 |

Ratio of active in pellets (A:B) = 93%:7%
Excipients in pellets A = 55.8%
Excipients in pellets B = 35.4%
Excipients except from pellets A&B = 46.6%

### Polymers

EUDRAGIT® NE and Eudragit® NM which are copolymers composed of free-radically polymerized units of 30% by weight of ethyl acrylate and 70% by weight of methyl methacrylate. EUDRAGIT® NE 30D is a 30% aqueous dispersion of EUDRAGIT® NE. EUDRAGIT® NM 30D is a 30% aqueous dispersion of EUDRAGIT® NM. EUDRAGIT® NM 30D respectively EUDRAGIT® NM is prepared according to WO 01/68767 and comprises a nonionic emulsifier having an HLB value of 15.2 to 17.3. In examples 1 to 5 EUDRAGIT® NE was used as a coating polymer.

### Innovator

Pellets A and B and compressed tablets made there from were compared to a so called “innovator” product, which is TOPROL® XL is an originator product of metoprolol succinate in the form of a compressed tablet containing 200 mg metoprolol succinate. A tablet of TOPROL® XL comprises a multitude of one type of metoprolol succinate pellets comprising metoprolol succinate containing cores and an extended release coating based on the polymer ethyl cellulose.

As interpreted from the innovator U.S. Pat. Nos. 4,957,745 and 4,927,640, earlier listed in Orange Book ‘OB_Rx’, the tablet is a multiparticulate system having pellets of one type containing insoluble inert silica core. On the core, metoprolol drug is coated by wurster process, finally overcoated with ethyl cellulose and hydroxypropylmethyl cellulose.

### Media

1. pH 6.8 phosphate buffer media as per USP 32

2. pH 1.2 HCl with 40% ethanol US Food and Drug Administration (FDA) and the Office of Generic Drugs (OGD) recommendations (Nonbinding Recommendations/Draft Guidance for Metoprolol Succinate, recommended January 2008, April 2008)

### Determination of the Metoprolol Release Values

| DISSOLUTION | | OGD test condition for release in |
|---|---|---|
| PARAMETERS | USP test conditions | 40% alcoholic 0.1N HCl pH 1.2 |
| Apparatus | USP-II (Paddle) | USP-II (Paddle) |
| Rpm | 50 | 50 |
| Temperature | 37° C. | 37° C. |
| Volume | 500 mL | 900 mL |
| Media | USP phosphate | 0.1N HCI substituting 40%v/v of |
| | buffer pH 6.8 | test medium with alcohol |
| Detection | 280 NM-HPLC | 280 NM-HPLC |
| Sampling points | 1, 4, 8 & 20 hrs | 15, 30, 45, 60, 75, 90, 105 and |
| | | 120 min |

### Limits:

1. For USP test Conditions, pH 6.8:
   Time

| Time (Hrs) | % Drug Dissolved |
|---|---|
| 1 | Not more than 25 |
| 4 | 20-40 |
| 8 | 40-80 |
| 20 | Not less than 80 |

2. For OGD test condition for release in 40% alcoholic 0.1N HCl
Release should be comparable or lower than the originator (RLD) in specified alcoholic media. In case of comparable release, f2 should be more than 50.

### Calculation of the Similarity Factor f2

The calculation of the similarity factor f2 is well known to a skilled person in the field of pharmacy or galenics. The similarity factor f2 may be calculated as described in “Guidance for Industry: Dissolution Testing of Immediate Release Solid Oral Dosage Forms. U.S Department of Health and Human Services, Food and Drug Administration, Center for Drug Evaluation and Research (CDER), August 1997”.

A simple model independent approach uses a difference factor (f₁) and a similarity factor ₁ (f 2) to compare dissolution profiles (Moore 1996). The difference factor (f₁) calculates the ₂₁ percent (%) difference between the two curves at each time point and is a measurement of the relative error between the two curves, where n is the number of time points, R is the dissolution value of the reference ₜ (prechange)

The similarity factor (f₂) is a logarithmic reciprocal square root transformation of the sum ₂ of squared error and is a measurement of the similarity in the percent (%) dissolution between the two curves.

$${f}_{2} = 50 log \{ {[ 1 + \frac{1}{n} {\sum }_{t = 1}^{n} {( {R}_{t} - {T}_{t} )}^{2} ]}^{\cdot 0.5} \times 100 \}$$

A specific procedure to determine difference and similarity factors is as follows:

1. Determine the dissolution profile of two products (12 units each) of the test (postchange) and reference (prechange) products.

2. Using the mean dissolution values from both curves at each time interval, calculate the difference factor (f1) and similarity factor (f2) using the above ₁₂ equations.

3. For curves to be considered similar, f values should be close to 0, and f₁₂ values should be close to 100. Generally, f 1 values up to 15 (0-15) and f 2 values greater than 50 (>50-100) ensure sameness or equivalence of the two curves and, thus, of the performance of the test (postchange) and reference (prechange) products. This model independent method is most suitable for dissolution profile comparison when three to four or more dissolution time points are available. As further suggestions for the general approach, the following recommendations should also be considered:
The dissolution measurements of the test and reference batches should be made under exactly the same conditions. The dissolution time points for both the profiles should be the same (e.g., 15, 30, 45, 60 minutes). The reference batch used should be the most recently manufactured prechange product.
Only one measurement should be considered after 85% dissolution of both the products.
To allow use of mean data, the percent coefficient of variation at the earlier time points (e.g., 15 minutes) should not be more than 20%, and at other time points should not be more than 10%.
The mean dissolution values for R can be derived either from (1) last prechange (reference) batch or (2) last two or more consecutively manufactured prechange batches.

### Overview about the Examples

| | | | Type of | Amount of the | Tablet or | Dissolution curve | |
|---|---|---|---|---|---|---|---|
| | | | Pellets | polymeric compound | pellet mixture | Lower or Higher | |
| | pH of | | (A/B) or | EUDRAGIT ® NE | (pellet A:B | than originator | |
| | the | Addition | Tablet | [%] by weight in | metoprolol | product in 40% | F₂-values in comparison to |
| Example | medium | ethanol | (T) | relation to the core | succinate ratio) | alcoholic media. | “innovator” |
| 1A | 1.2 | 40% | A | 10/15/25/30/40 | | —/higher/higher/higher/lower | —/27.78/45.66/56.91/74.67 |
| 1B | 1.2 | — | A | 10/15/25/30/40 | | | —/61.98/64.12/46.41/46.85 |
| 1C | 6.8 | — | A | 20/25/30/40/43 | | | |
| 2 | 6.8 | — | B | 10 | | | |
| 3A | 1.2 | 40% | T | 43/10 | 70:30 | Lower | 36.55 |
| 3B | 1.2 | 40% | T | 43/10 | 85:15 | Lower | 38.84 |
| 3C | 1.2 | 40% | T | 43/10 | 93:7 | Lower | 54.72 |
| 4A | 6.8 | — | A | 43 | | | |
| 4B | 6.8 | — | B | 10 | | | |
| 4C | 6.8 | — | A + B | 43/10 | 93:7 | | |
| 5A | 6.8 | — | T | 43/10 | 70:30 | | |
| 5B | 6.8 | — | T | 43/10 | 85:15 | | |
| 5C | 6.8 | — | T | 43/10 | 93:7 | | |

### Summary of the Results of the Examples

### Example 1A

Pellets with 30 or 40% coating (EUDRAGIT® NE content coating/core) fulfil the 15 and 30 min release criteria as disclosed in here.

### Example 1B

Pellets with 15 or 25% coating (EUDRAGIT® NE content coating/core) show a comparable release rate to the innovator product.

### Example 1C

Pellets with 20 or 25% coating (EUDRAGIT® NE content coating/core) fulfil the 1 and 20 h pH 6.8 release criteria for metoprolol. Pellets with 30, 40 or 43% coating (EUDRAGIT® NE content coating/core) show the desired release rate of less than 40% after 4 h for pellet type A.

### Example 2

Pellets B (10% coated) show the desired release rate of more than 40% after 4 h.

### Example 3A-C

Tablets according to the invention show comparable or lower release profile than the innovator as defined under limits. Tablets in example 3A and 3B show lower release profile than the innovator product. In example 3C a similarity factor f2 is more than 50.

### Example 4A-C

Pellets A and B fulfil the desired release rate of less than 20%/more than 40% after 4 h as disclosed herein. In example 4C the mixed pellets A and B fulfil the 1, 4, 8 and 20 h pH 6.8 release criteria for metoprolol.

### Example 5A-C

All three tablets with different pellet A/B ratios fulfil the 1, 4, 8 and 20 h pH 6.8 release criteria for metoprolol.

### Examples 1A-C

Example 1A

| | | | Amount of EUDRAGIT ® NE [%] in relation to core by weight. | | | | |
|---|---|---|---|---|---|---|---|
| Metoprolol release | Time | Innovator | 10% | 15% | 25% | 30% | 40% |
| [%] Pellets | (min.) | Mean | Mean | Mean | Mean | Mean | Mean |
| In 0.1N HCl pH 1.2 + | 0.00 | 0.00 | 0.00 | 0.00 | 0.00 | 0.00 | 0.00 |
| 40% Ethanol | 15.0 | 4.77 | 60.02 | 29.64 | 19.79 | 9.70 | 5.98 |
| | 30.0 | 24.02 | 82.34 | 61.04 | 45.67 | 36.10 | 27.49 |
| | 45.0 | 52.59 | 90.86 | 79.94 | 63.86 | 59.45 | 50.56 |
| | 60.0 | 69.68 | 94.75 | 88.53 | 75.41 | 74.88 | 66.66 |
| | 75.0 | 79.08 | 96.69 | 94.74 | 82.56 | 86.31 | 76.36 |
| | 90.0 | 84.88 | 99.56 | 98.65 | 89.54 | 88.75 | 82.03 |
| | 105.0 | 90.06 | 100.49 | 99.43 | 93.64 | 91.27 | 85.85 |
| | 120.0 | 91.50 | 101.68 | 99.86 | 96.95 | 92.86 | 87.69 |
| | F₂ value | | | 27.78 | 45.66 | 56.91 | 74.67 |

Example 1B

| | | | Amount of EUDRAGIT ® NE [%] in relation to core by weight. | | | | |
|---|---|---|---|---|---|---|---|
| Metoprolol release | Time | Innovator | 10% | 15% | 25% | 30% | 40% |
| [%] Pellets | (min.) | Mean | Mean | Mean | Mean | Mean | Mean |
| In 0.1N HCl pH 1.2 | 0.00 | 0.00 | 0.00 | 0.00 | 0.00 | 0.00 | 0.00 |
| | 15.0 | 3.95 | 3.83 | 1.04 | 0.62 | 0.28 | 0.25 |
| | 30.0 | 7.46 | 7.98 | 2.58 | 1.55 | 0.59 | 0.51 |
| | 45.0 | 10.26 | 13.46 | 4.20 | 3.40 | 0.98 | 0.88 |
| | 60.0 | 12.66 | 19.98 | 5.92 | 5.69 | 1.43 | 1.39 |
| | 75.0 | 14.77 | 26.68 | 7.91 | 8.60 | 1.97 | 2.06 |
| | 90.0 | 16.73 | 33.26 | 10.21 | 11.96 | 2.68 | 2.94 |
| | 105.0 | 18.49 | 39.56 | 12.80 | 15.76 | 3.53 | 4.03 |
| | 120.0 | 20.26 | 45.69 | 15.67 | 19.95 | 4.54 | 5.30 |
| | F₂ value | | | 61.98 | 64.12 | 46.41 | 46.85 |

Example 1C

| Metoprolol release | | | Amount of EUDRAGIT ® NE [%] in relation to core by weight. | | | | |
|---|---|---|---|---|---|---|---|
| [%] Pellets In pH | Time | Innovator | 20% | 25% | 30% | 40% | 43% |
| 6.8 buffer | (Hr.) | Mean | Mean | Mean | Mean | Mean | Mean |
| | 0 | 0.0 | 0.0 | 0.0 | 0.00 | 0.0 | 0.0 |
| | 1 | 9.0 | 12.4 | 4.7 | 0.95 | 0.9 | 1.0 |
| | 2 | 15.4 | 31.1 | 15.5 | 3.83 | 3.6 | 3.5 |
| | 4 | 27.4 | 58.0 | 40.8 | 15.21 | 14.0 | 13.0 |
| | 6 | 39.9 | 75.7 | 60.7 | 30.26 | 28.5 | 26.7 |
| | 8 | 51.6 | 86.5 | 74.8 | 44.56 | 42.5 | 40.1 |
| | 10 | 63.5 | 93.3 | 84.7 | 57.09 | 54.5 | 51.3 |
| | 12 | 73.6 | 97.3 | 90.7 | 68.15 | 64.7 | 60.7 |
| | 14 | 81.1 | 99.9 | 94.6 | 76.64 | 72.8 | 68.3 |
| | 16 | 87.0 | 101.4 | 97.0 | 82.74 | 79.2 | 74.3 |
| | 18 | 90.7 | 102.6 | 98.9 | 87.62 | 84.2 | 79.2 |
| | 20 | 93.7 | 103.5 | 100.2 | 91.34 | 88.3 | 83.3 |
| | 24 | 97.8 | 105.1 | 101.6 | 95.85 | 94.2 | 88.8 |

### Example 2

Metoprolol release [%] of pellets B (10% coated) in ph 6.8 buffer medium

| | Batch 1 | Batch 2 | Batch 3 |
|---|---|---|---|
| Time | % Drug | % Drug | % Drug |
| (Hr.) | released | released | released |
| 0 | 0.00 | 0.00 | 0.00 |
| 1 | 18.70 | 14.21 | 23.68 |
| 2 | 40.69 | 32.50 | 47.57 |
| 4 | 70.12 | 63.11 | 76.54 |
| 8 | 92.74 | 92.28 | 96.01 |
| 12 | 97.99 | 99.35 | 100.95 |
| 16 | 99.88 | 101.55 | 102.39 |
| 20 | 100.60 | 102.89 | 103.08 |
| 24 | 101.75 | 103.65 | 103.36 |

### Examples 3A-C

Metoprolol release [%] tablets with different pellet A: B metoprolol succinate ratios by weight in 0.1N HCl pH 1.2+40% Ethanol compared to the “innovator” product.

### Example 3A

| | Tablet (15Pellet B: | |
|---|---|---|
| Time | 85Pellet A) | Innovator |
| (min.) | % Drug Release | % Drug Release |
| 0 | 0.00 | 0.00 |
| 15 | 6.53 | 6.36 |
| 30 | 17.93 | 30.10 |
| 45 | 32.89 | 58.26 |
| 60 | 46.93 | 72.86 |
| 75 | 58.13 | 80.73 |
| 90 | 66.30 | 85.38 |
| 105 | 72.76 | 88.36 |
| 120 | 77.52 | 90.58 |

| | |
|---|---|
| F₂ value | 36.55 |
| | (The profile is lower as compared to |
| | Innovator) |

### Example 3B

| | Tablet | |
|---|---|---|
| | (30Pellet B: | |
| Time | 70PelletA) | Innovator |
| (min.) | % Drug Release | % Drug Release |
| 0.00 | 0.00 | 0.00 |
| 15.0 | 10.65 | 6.36 |
| 30.0 | 24.12 | 30.10 |
| 45.0 | 38.25 | 58.26 |
| 60.0 | 50.22 | 72.86 |
| 75.0 | 59.58 | 80.73 |
| 90.0 | 66.61 | 85.38 |
| 105.0 | 72.13 | 88.36 |
| 120.0 | 76.51 | 90.58 |

| | |
|---|---|
| F₂ value | 38.84 |
| | (The release profile is lower as compared |
| | to Innovator) |

### Example 3C

| | Tablet | |
|---|---|---|
| | (7Pellet B: | |
| Time | 93PelletA) | Innovator |
| (min.) | % Drug Release | % Drug Release |
| 0.00 | 0.00 | 0.00 |
| 15.0 | 6.86 | 6.36 |
| 30.0 | 24.99 | 30.10 |
| 45.0 | 45.52 | 58.26 |
| 60.0 | 59.55 | 72.86 |
| 75.0 | 72.09 | 80.73 |
| 90.0 | 78.92 | 85.38 |
| 105.0 | 83.99 | 88.36 |
| 120.0 | 87.64 | 90.58 |

| | |
|---|---|
| F₂ value | 54.72 |
| | (The release profile is lower as compared |
| | to Innovator) |

### Example 4A-C

Dissolution profile of mixture of pellets (ratio by metoprolol weight 7Pellet B:93PelletA) in comparison to pellets A and B alone in pH 6.8 buffer medium

| | Example 4C | Example 4B | Example 4A |
|---|---|---|---|
| | Mix pellets | Pellet B | Pellet A |
| Time in Hr. | % Drug Release | % Drug Release | % Drug Release |
| 0.0 | 0 | 0 | 0 |
| 1.0 | 3 | 19 | 1 |
| 2.0 | 7 | 41 | 4 |
| 4.0 | 20 | 70 | 14 |
| 8.0 | 47 | 93 | 42 |
| 12.0 | 66 | 98 | 64 |
| 16.0 | 79 | 100 | 78 |
| 20.0 | 86 | 101 | 86 |
| 24.0 | 91 | 102 | 91 |

### Example 5A-C

### Dissolution Profile in pH 6.8 Buffer Medium

of compressed tablets containing different pellets A:B mixtures with the given metoprolol ratios by weight

| Time | Example 5A: Tablet | Example 5B: Tablet | Example 5C: Tablet |
|---|---|---|---|
| in | (30Pellet B:70 | (15Pellet B:85 | (7Pellet B:93 |
| | PelletA) | PelletA) | PelletA) |
| Hr. | % Drug Release | % Drug Release | % Drug Release |
| 0 | 0.0 | 0.0 | 0.0 |
| 1 | 10.7 | 5.4 | 5.9 |
| 2 | 21.0 | 11.8 | 13.1 |
| 4 | 37.2 | 25.7 | 28.1 |
| 8 | 58.7 | 50.1 | 53.7 |
| 12 | 72.8 | 68.0 | 71.6 |
| 16 | 82.0 | 80.4 | 83.0 |
| 20 | 88.1 | 89.6 | 89.0 |
| 24 | 92.0 | 94.7 | 93.0 |

1. A multiparticulate pharmaceutical composition comprising a multitude of pellets A and pellets B, each of the pellets A and the pellets B comprising metoprolol or a pharmaceutically acceptable salt thereof as an active pharmaceutical ingredient,
wherein the pellets A are coated with a first coating layer that is in an amount sufficient to result in an active pharmaceutical ingredient release profile according to USP in pH 6.8 test medium with a release rate of less than 20% after 4 hours, the first coating layer comprising at least 30% by weight of a polymeric compound, and the polymeric compound consisting of one or more (meth)acrylate copolymers polymerized from 20% to 40% by weight of ethyl acrylate, 60% to 80% by weight of methyl methacrylate, and 0% or less than 5% by weight of methacrylic acid or acrylic acid,
wherein the pellets B are optionally coated with a second coating layer and show an active pharmaceutical ingredient release profile according to USP in pH 6.8 test medium with an active pharmaceutical ingredient release rate of more than 40% after 4 hours,
wherein the pellets A have a metoprolol release rate, in a pH 1.2 test medium according to USP with addition of 40% (v/v) ethanol, of
not more than 15% after 15 minutes
more than 15% and up to 40% after 30 minutes,
wherein the pellets B have a metoprolol release rate, in a pH 1.2 test medium according to USP with addition of 40% (v/v) ethanol, of
more than 15% after 15 minutes
more than 40% after 30 minutes
and wherein the pellets A and the pellets B are present in the multiparticulate pharmaceutical composition in a relation that results in a combined active pharmaceutical ingredient release profile of the multiparticulate pharmaceutical composition according to USP in a pH 6.8 test medium with release rates of
not more than 25% after 1 hour
20% to 40% after 4 hours
40% to 60% after 8 hours
not less than 80% after 20 hours.

2. The multiparticulate pharmaceutical composition according to claim 1, wherein the polymeric compound is present in an amount of at least 28% by weight based on the weight of pellet A cores.

3. The multiparticulate pharmaceutical composition according to claim 1, wherein the pellets comprise at least 65% by weight of the active pharmaceutical ingredient and the pellets B comprise not more than 35% by weight of the active pharmaceutical ingredient.

4. The multiparticulate pharmaceutical composition according to claim 1, wherein the metoprolol salt is a benzoate salt, a fumarate salt, a succinate salt or a tartrate salt.

5. The multiparticulate pharmaceutical composition according to claim 1, wherein the metoprolol is present in an amount of 5% to 75% by weight based on a total weight of the multiparticulate pharmaceutical composition.

6. The multiparticulate pharmaceutical composition according to claim 1, wherein the pellets A further comprise up to 70% by weight of one or more pharmaceutically acceptable excipients, based on a total weight of the multiparticulate pharmaceutical composition.

7. The multiparticulate pharmaceutical composition according to claim 1, wherein the pellets B are coated with the second coating layer, the second coating layer comprising at least 30% by weight of one or more (meth)acrylate copolymers polymerized from 20% to 40% by weight of ethyl acrylate, 60% to 80% by weight of methyl methacrylate and 0% or less than 5% by weight of methacrylic acid or acrylic acid.

8. The multiparticulate pharmaceutical composition according to claim 1, wherein the pellets B further comprise up to 50% by weight of one or more pharmaceutically acceptable excipients, based on a total weight of the multiparticulate pharmaceutical composition.

9. The multiparticulate pharmaceutical composition according to claim 1, wherein the multiparticulate pharmaceutical composition is in the form of a compressed tablet, a sachet or a capsule.

10. The multiparticulate pharmaceutical composition according to claim 1, comprising at least 30% by weight of the pellets A and the pellets B.

11. A process for preparing the multiparticulate pharmaceutical composition according to claim 1, comprising:
extruding and spheronizing an active pharmaceutical ingredient comprising uncoated pre-pellets A and uncoated pre-pellets B,
coating the uncoated pre-pellets A and optionally the uncoated pre-pellets B by spray coating, and
formulating pellets A and pellets B as the multiparticulate pharmaceutical composition by compression into tablets, by formulation of sachets or by filling into capsules.
